(19) United States
(12) Patent Application Publication
(10) Pub. No.: US 2020/0350060 A1
(43) Pub. Date: Nov. 5, 2020

(54) METHOD AND SYSTEM FOR DELIVERING MEDICAL CARE TO PATIENTS

(71) Applicant: Nicholas Bastidas, New York, NY (US)

(72) Inventor: Nicholas Bastidas, New York, NY (US)

(21) Appl. No.: 16/399,345

(22) Filed: Apr. 30, 2019

(51) Int. Cl. G16H 40/20; G16H 80/00

(52) U.S. Cl. CPC G16H 40/20; G16H 80/00

(57) ABSTRACT

A method for delivering medical care to a patient is provided. A first set of doctors is identified for performing a medical procedure on the patient based on a first location of the patient. An availability request is communicated to a subset of the first set of doctors, requesting an availability confirmation of each doctor of the subset of doctors for performing the medical procedure. One or more availability responses of one or more doctors are received from one or more doctor devices. Each availability response is indicative of an availability of a corresponding doctor for performing the medical procedure. A first doctor is selected, from the one or more doctors, for performing the medical procedure. The first doctor is selected based on a response time of the availability response of the first doctor to the availability request.

### FIELD OF THE INVENTION

Various embodiments of the present invention relate generally to healthcare systems. More specifically, various embodiments of the present invention relate to a method and a system for delivering medical care to a patient.

### BACKGROUND

Patients in need of immediate medical attention are often taken to medical facilities (such as clinics and/or hospitals) for treatment. In these medical facilities, the patients may be subjected to long waiting periods prior to treatment. These long waiting periods may be caused due to a variety of reasons, for example, unavailability of doctors to treat the patients, large number of patients requiring emergency treatment, or the like. Such long waiting periods may be detrimental to treatment and recovery of the patients. Additionally, the patients may not be proffered any choice to select doctors of their choosing. For example, a doctor allocated to a patient by a medical facility may not specialize in a kind of treatment required by the patient. The patient may have little to no awareness of the credentials (e.g., timespan of medical experience or area of specialization) of the doctor allocated to the patient. In case of emergencies, patients may not have the luxury of postponing treatment and/or scheduling appointments with desired doctors (i.e., specialists). For example, a first patient with lacerations on a face of the first patient may be allocated a physician, and not a plastic surgeon, for treating the lacerations. If the physician is not trained to treat complex wounds on the face, a result of an operation performed by the physician to repair the lacerations on the face may be sub-par, resulting in permanent scarring on the face of the first patient. Current solutions allow patients to schedule appointments online with doctors for consultations. However, these solutions do not permit patients to schedule surgeries or other invasive procedures. Further, these solutions may not prove useful in emergency situations where patients require immediate medical care.

In light of the foregoing, there is a need for a technical and reliable solution that solves the above-mentioned problems and facilitates efficient delivery of medical care to a patient in case of emergencies.

### SUMMARY

In an embodiment of the present invention, a method for delivering medical care to a patient is provided. The method includes identifying, by a server, a first set of doctors for performing a medical procedure on the patient, based on a first location of the patient. An availability request is communicated, by the server, to a subset of the first set of doctors, requesting an availability confirmation of each doctor of the subset of doctors for performing the medical procedure. One or more availability responses of one or more doctors are received by the server from one or more doctor devices. Each availability response is indicative of an availability of a corresponding doctor for performing the medical procedure. The subset of doctors comprises the one or more doctors. A first doctor is selected by the server from the one or more doctors, for performing the medical procedure. The first doctor is selected based on a response time of the availability response of the first doctor to the availability request.

In another embodiment of the present invention, a system for delivering medical care to a patient is provided. The system includes a server that is configured to identify, based on a first location of a patient, a first set of doctors for performing a medical procedure on the patient. The server communicates an availability request to a subset of the first set of doctors, requesting an availability confirmation of each doctor of the subset of doctors for performing the medical procedure. The server receives from, from one or more doctor devices, one or more availability responses of one or more doctors. Each availability response is indicative of an availability of a corresponding doctor for performing the medical procedure. The subset of doctors comprises the one or more doctors. The server selects a first doctor from the one or more doctors, for performing the medical procedure. The first doctor is selected based on a response time of the availability response of the first doctor to the availability request.

### BRIEF DESCRIPTION OF THE DRAWINGS

Various embodiments of the present invention are illustrated by way of example, and not limited by the appended figures, in which like references indicate similar elements, and in which:

FIG. 1 is a block diagram that illustrates an exemplary environment for delivering medical care to a patient, in accordance with an embodiment of the present invention;

FIG. 2 represents a process flow diagram that illustrates an exemplary scenario for creating a first patient profile of a patient with a healthcare server of FIG. 1, in accordance with an embodiment of the present invention;

FIG. 3 represents a process flow diagram that illustrates an exemplary scenario for creating a first doctor profile of a doctor with the healthcare server, in accordance with an embodiment of the present invention;

FIG. 4 is a Table that illustrates doctor profiles stored in a memory of the healthcare server, in accordance with an embodiment of the invention;

FIGS. 5A and 5B, collectively represent a process flow diagram that illustrates a method for delivering medical care to the patient, in accordance with an embodiment of the present invention;

FIGS. 6A and 6B, collectively represent a process flow diagram that illustrates a method for delivering medical care to the patient, in accordance with another embodiment of the present invention;

FIG. 7 represents an exemplary scenario that illustrates user interface (UI) screens rendered by a service application, in accordance with an embodiment of the present invention;

FIG. 8 is a block diagram that illustrates the healthcare server, in accordance with an embodiment of the present invention; and

FIG. 9 is block diagram that illustrates system architecture of a computer system, in accordance with an embodiment of the present invention; and

FIGS. 10A-10C, collectively represent a flow chart that illustrates a method for delivering medical care to the patient, in accordance with an embodiment of the present invention.

Further areas of applicability of the present invention will become apparent from the detailed description provided hereinafter. It should be understood that the detailed description of exemplary embodiments is intended for illustration purposes only and is, therefore, not intended to necessarily limit the scope of the present invention.

### DETAILED DESCRIPTION

The present invention is best understood with reference to the detailed figures and description set forth herein. Various embodiments are discussed below with reference to the figures. However, those skilled in the art will readily appreciate that the detailed descriptions given herein with respect to the figures are simply for explanatory purposes as the methods and systems may extend beyond the described embodiments. In one example, the teachings presented and the needs of a particular application may yield multiple alternate and suitable approaches to implement the functionality of any detail described herein. Therefore, any approach may extend beyond the particular implementation choices in the following embodiments that are described and shown.

References to “an embodiment”, “another embodiment”, “yet another embodiment”, “one example”, “another example”, “yet another example”, “for example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment.

### Overview

A patient in need of immediate medical attention may be taken to a medical facility (such as a clinic or hospital) for treatment. The patient may be subjected to a long waiting period prior to treatment. Further, the patient may not be aware of the credentials of a doctor allocated by the medical facility for the treatment of the patient. The patient may have no say in the allocation of the doctor to the patient. Thus, the patient may receive sub-optimal treatment and/or inadequate medical attention.

Various embodiments of the present invention provide a method and a system that solve the abovementioned problems by enabling the patient to select doctors for treatment of medical conditions, in case of medical emergencies. A server may receive, from a patient device of the patient, a request for performing a medical procedure on the patient. The request may indicate a set of preferences of the patient. Based on the set of preferences and a location of the patient, the server may identify a first set of doctors for performing a medical procedure on the patient. The server may communicate a message to a subset of the first set of doctors, requesting an availability confirmation from each doctor of the subset of doctors. The server may receive one or more availability responses from one or more doctors of the subset of doctors, by way of doctor devices of the one or more doctors. Each availability response is indicative of an availability of a corresponding doctor for performing the medical procedure. The server selects, from the one or more doctors, a first doctor for performing the medical procedure. The first doctor may be selected based on a response time of the availability response of the first doctor to the message. Based on the selection of the first doctor, the server may communicate, to the patient device, a notification indicating the selection of the first doctor for performing the medical procedure. The server may further communicate patient details of the patient to the first doctor based on the selection of the first doctor. The patient details may be indicative of a name, contact information, and a medical condition of the patient. Using the patient details, the first doctor may contact the patient for arranging an appointment with the patient.

Thus, the method and system of the present invention enables the patient to select doctors for treatment of the medical condition of the patient based on the set of preferences of the patient.

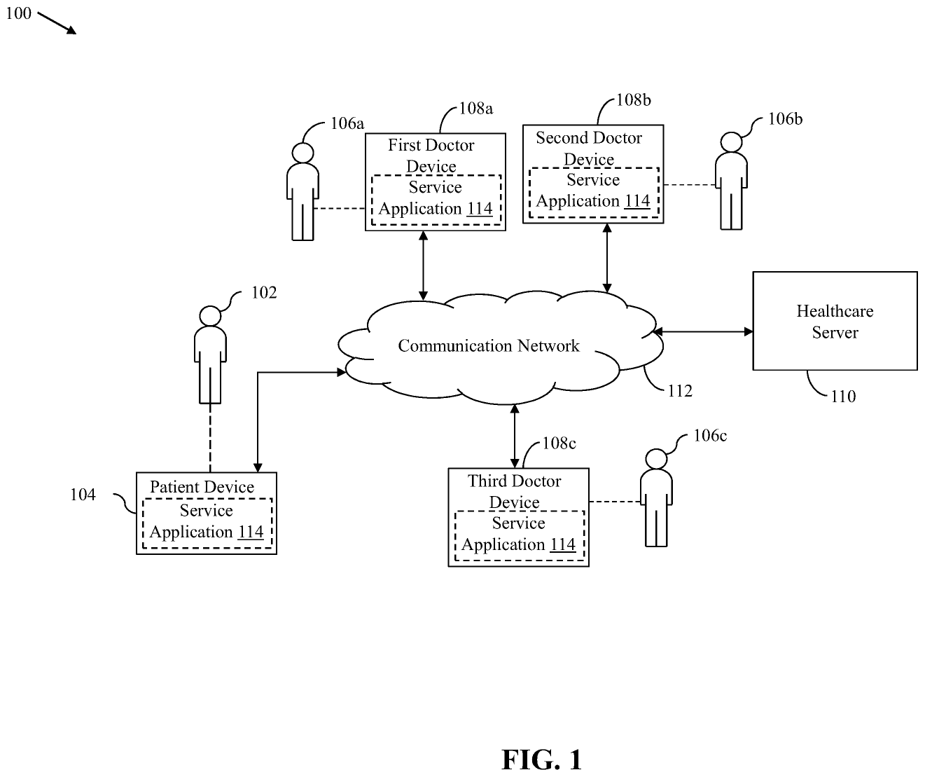

FIG. 1 is a block diagram that illustrates an exemplary environment 100 for delivering medical care to a patient, in accordance with an embodiment of the present invention. The environment 100 includes a patient 102 in possession of a patient device 104, a first doctor 106a in possession of a first doctor device 108a, a second doctor 106b in possession of a second doctor device 108b, and a third doctor 106c in possession of a third doctor device 108c. The environment 100 further includes a healthcare server 110. Hereinafter, the first through third doctors 106a-106c and the first through third doctor devices 108a-108c are collectively referred to and designated as “the doctors 106” and the “the doctor devices 108”, respectively. The patient device 104, the doctor devices 108, and the healthcare server 110 may communicate with each other by way of a communication network 112 or through separate communication networks established therebetween.

The patient 102 is an individual, who may have a first medical condition and may require medical care or medical assistance for treating the first medical condition. Examples of the first medical condition may include, but are not be limited to, a physical medical condition (e.g., a viral and/or bacterial infection, a laceration, a burn, a fracture, or the like) or a mental medical condition (e.g., depression, or the like).

The patient device 104 is a communication device of the patient 102. Examples of the patient device 104 may include a smartphone, a personal computer, a tablet, a phablet, or the like. The patient device 104 may be used by the patient 102 to access a service application 114 for selecting a suitable doctor to treat the first medical condition of the patient 102. The service application 114 may be a mobile application or a web application that runs or is executed on the patient device 104.

The doctors 106 are medical practitioners who are registered with the healthcare server 110 for providing medical care to patients, such as the patient 102. In other words, the doctors 106 are individuals who are certified for performing medical procedures on the patients in a corresponding field of expertise. Each doctor 106 may work at a hospital and/or may be engaged in private practice. Each doctor 106 may have specialized in one or more medical fields (for example, plastic surgery, oncology, radiology, ophthalmology, or the like) and may be capable of treating physical and/or mental medical conditions of the patients as per the medical specialization. For example, the first doctor 106a may be a plastic surgeon, who is capable of performing invasive or non-invasive medical procedures.

The doctor devices 108 are communication devices of the doctors 106. It will be apparent to those of skill in the art that the doctor devices 108 are functionally similar to the patient device 104. The doctor devices 108 may be configured to execute the service application 114. The service application 114 that runs on each of the doctor devices 108 may be utilized by the corresponding doctor 106 for delivering medical care to the patients. For example, the first doctor 106a may access the service application 114 that runs on the first doctor device 108a for responding to requests (e.g., a request initiated by the patient 102) from patients and contacting the patients associated with the requests for delivering medical care to the patients. Examples of the doctor devices 108 may include smartphones, personal computers, tablets, phablets, or the like.

The healthcare server 110 may include suitable logic, circuitry, interfaces and/or code, executable by the circuitry, that may be configured to perform one or more activities for delivering medical care to the patients (e.g., the patient 102). In other words, the healthcare server 110 offers and provides a medical care service to the patients. In one example, the healthcare server 110 is a computing server associated with a healthcare entity for a purpose of delivering medical care to the patients. In a non-limiting example, the healthcare entity may be an aggregator of medical practitioners (such as the doctors 106). In another example, the healthcare entity may be a medical association or a consortium of hospitals and/or clinics. In a non-limiting example, it is assumed that three doctors (i.e., the doctors 106) have registered with the healthcare entity for the purpose of delivering medical care to the patients. It will be apparent to those of skill in the art that any number of doctors may be associated with the healthcare entity without deviating from the scope of the invention.

The healthcare server 110 may be configured to manage and maintain doctor profiles of registered doctors (e.g., the doctors 106) and patient profiles of patients (e.g., the patient 102), who have signed up for availing the medical care service. For example, the healthcare server 110 may store first through third doctor profiles of the doctors 106, respectively, and a first patient profile of the patient 102 in a memory of the healthcare server 110. The first doctor profile of the first doctor 106a may indicate details such as, but not limited to, a timespan of medical experience of the first doctor 106a, medical qualifications of the first doctor 106a, a field of specialization of the first doctor 106a, a review rating of the first doctor 106a, or the like. Likewise, the first patient profile of the patient 102 may indicate details such as, but not limited to, personal details (such as a name, an age, a gender, contact details, or the like) of the patient 102 and medical details (such as a name of an insurance provider, an insurance policy identifier of an insurance policy, a policy coverage amount of the insurance policy, a medical history of the patient 102, or the like) of the patient 102. The healthcare server 110 may be realized through various web-based technologies, such as, but not limited to, a Java web-framework, a .NET framework, a PHP (Hypertext Preprocessor) framework, or the like. Examples of the healthcare server 110 may include, but are not limited to, a personal computer, a laptop, or a network of computer systems. In a non-limiting example, the healthcare server 110 is a health insurance portability and accountability act (HIPAA) compliant server. Thus, the patient and doctor profiles are stored safely and securely in the memory of the healthcare server 110.

The healthcare server 110 may be configured to host the service application 114. The service application 114 is executable on various patient devices (such as the patient device 104) and various doctor devices (such as the first doctor device 108a). The service application 114 may allow the patients (e.g., the patient 102) to initiate requests for medical care and to select doctors for delivering medical care. The service application 114 may further allow doctors (such as the doctors 106) to respond to the requests of the patients. In one embodiment, the healthcare server 110 may be configured to select a suitable doctor for a patient (such as the patient 102) based on a request of the patient 102 and then establish a real-time electronic communication link between the patient device 104 and a doctor device of the selected doctor.

The communication network 112 is a medium through which content and messages are transmitted between the patient device 104, the doctor devices 108, and the healthcare server 110. Examples of the communication network 112 include, but are not limited to, a Wi-Fi network, a light fidelity (Li-Fi) network, a local area network (LAN), a wide area network (WAN), a metropolitan area network (MAN), a satellite network, the Internet, a fiber optic network, a coaxial cable network, an infrared (IR) network, a radio frequency (RF) network, and combinations thereof. Various entities in the environment 100 may connect to the communication network 112 in accordance with various wired and wireless communication protocols, such as Transmission Control Protocol and Internet Protocol (TCP/IP), User Datagram Protocol (UDP), Long Term Evolution (LTE) communication protocols, or any combination thereof.

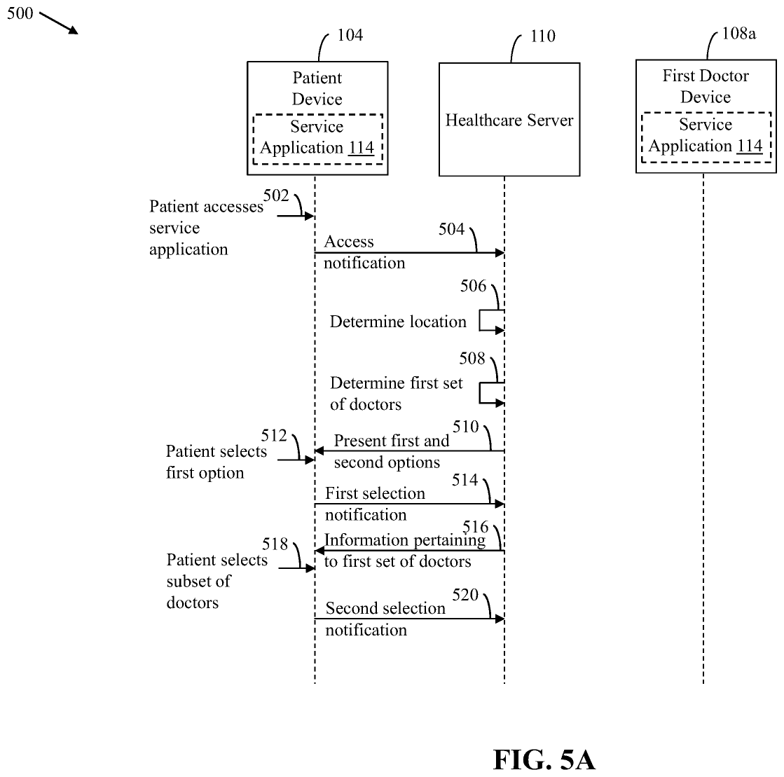

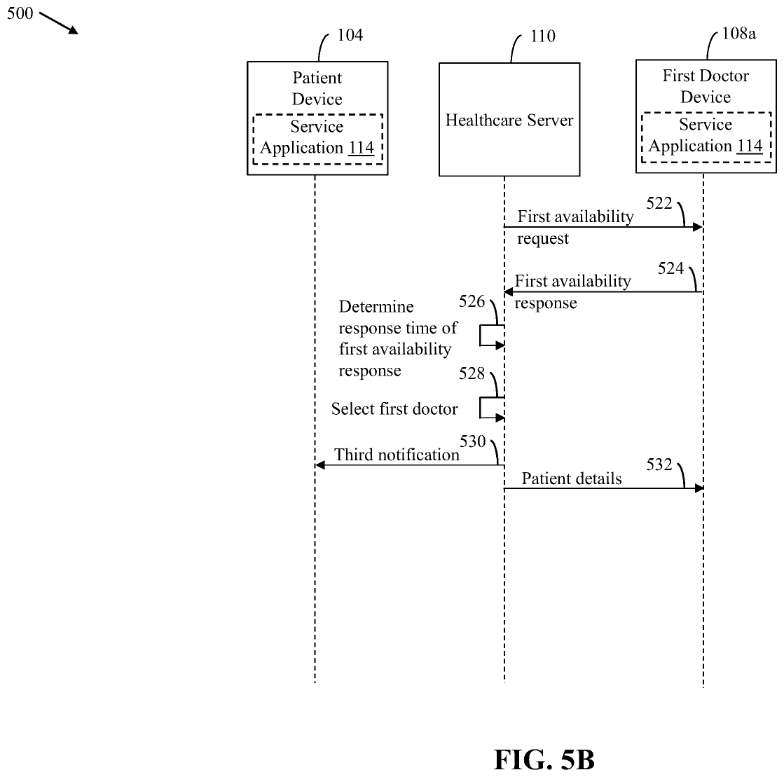

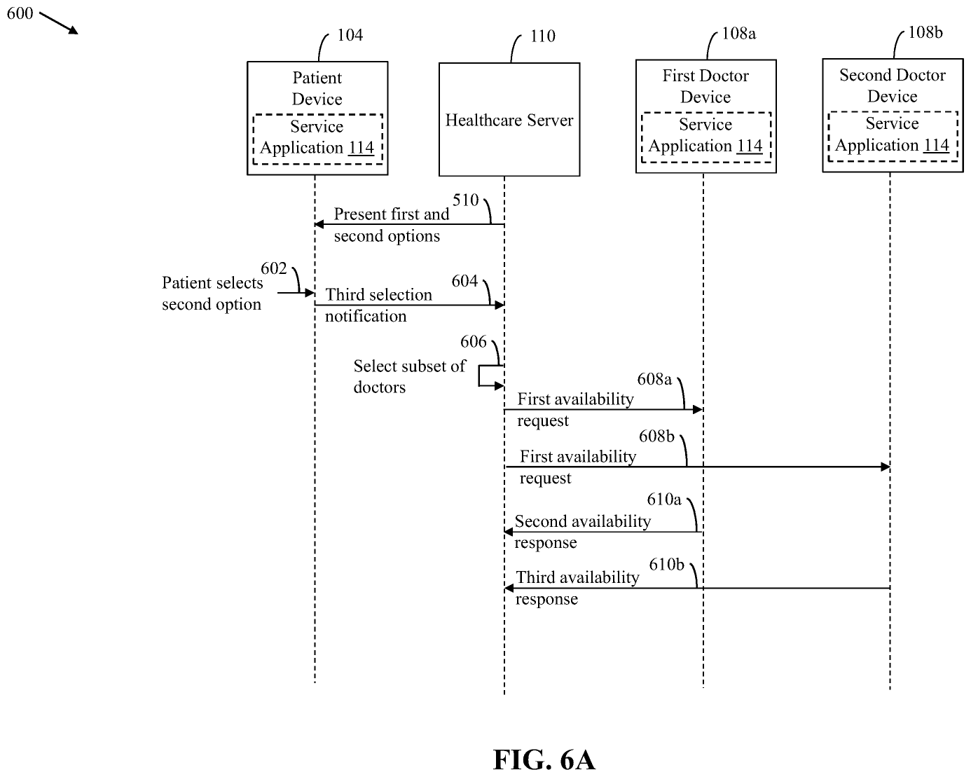

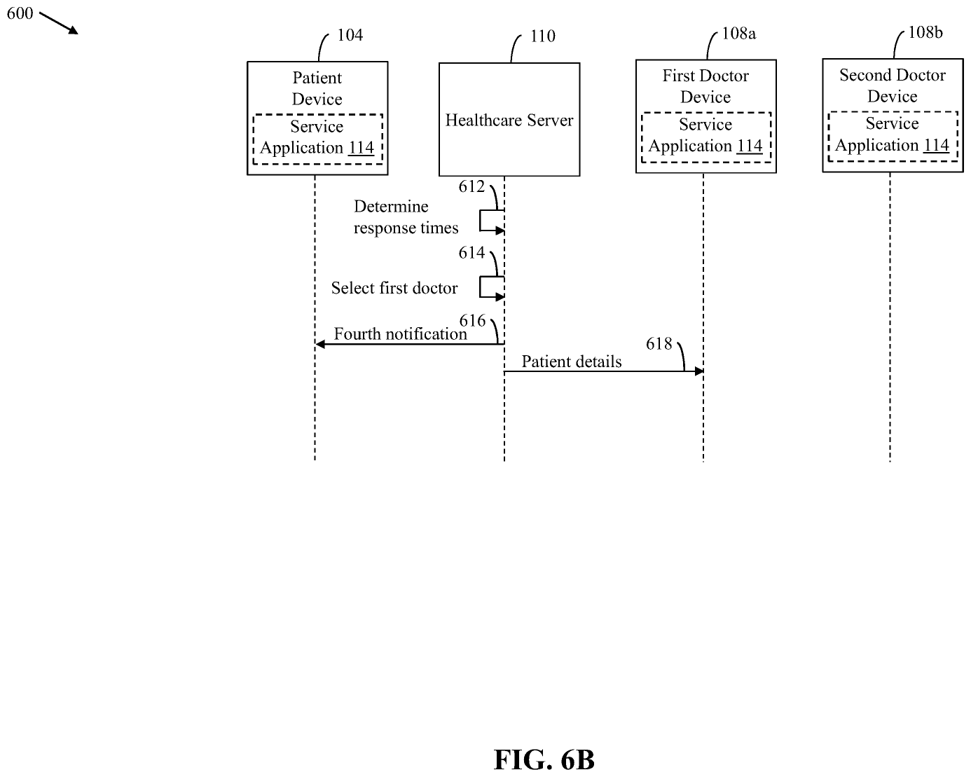

In operation, the patient 102 may be suffering from the first medical condition, for example, a fracture in an elbow. The patient device 104 may be utilized by the patient 102 to select a suitable doctor for performing a first medical procedure to treat the first medical condition. The patient 102 may access the service application 114 that runs or is executed on the patient device 104 for selecting the suitable doctor. When the patient 102 performs a first activity on the patient device 104 (i.e., accesses the service application 114), the patient device 104 communicates an access notification to the healthcare server 110. Based on the access notification, the healthcare server 110 determines a location of the patient device 104 (i.e., the location of the patient 102). The healthcare server 110 then determines a first set of doctors that are available for performing the first medical procedure to treat the first medical condition of the patient 102. The healthcare server 110 further presents, to the patient 102, a list indicative of the first set of doctors by way of the service application 114. In one embodiment, the patient 102 may select a first subset of doctors from the first set of doctors for the performing the first medical procedure. In another embodiment, the healthcare server 110 may automatically select the first subset of doctors for the performing the first medical procedure. Based on the selection of the first subset of doctors, the healthcare server 110 may communicate a message to a doctor device of each doctor in the first subset of doctors, requesting the first subset of doctors to confirm an availability for performing the first medical procedure. Based on availability responses received from the doctor devices of the first subset of doctors and a response time of each availability response, the healthcare server 110 may select a suitable doctor (e.g., the first doctor 106a) for performing the first medical procedure. For example, the healthcare server 110 may select the first doctor 106a for performing the first medical procedure if a response time of an availability response of the first doctor 106a is less than the response times of the availability responses of all other doctors in the first subset of doctors. The healthcare server 110 then communicates the patient details of the patient 102 to the selected doctor (e.g., the first doctor 106a) and establishes a real-time electronic communication link between the patient device 104 and the first doctor device 108a of the first doctor 106a. Operations performed by the healthcare server 110 for providing the medical care service are explained in detail in conjunction with FIGS. 5A and 5B and 6A and 6B.

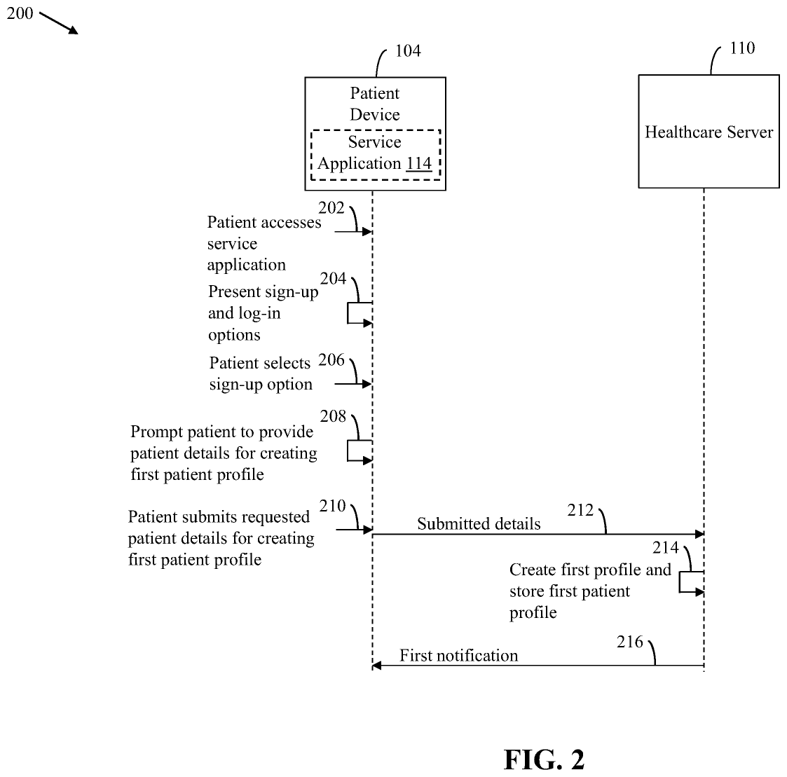

FIG. 2 represents a process flow diagram 200 that illustrates an exemplary scenario for creating the first patient profile of the patient 102 with the healthcare server 110, in accordance with an embodiment of the present invention. The process flow diagram 200 involves the patient device 104 and the healthcare server 110.

The patient device 104 may be utilized by the patient 102 to access the service application 114 that runs on the patient device 104 (as shown by arrow 202). The service application 114 may render a first user interface (UI) on a display of the patient device 104. The first UI may present a first sign-up option and a first log-in option to the patient 102 (as shown by arrow 204). The first sign-up option may allow the patient 102 to create a new patient profile and the log-in option may allow the patient 102 to log into an existing patient profile. In a non-limiting example, it is assumed that the patient 102 is a new user. Thus, the patient 102 selects the first sign-up option (as shown by arrow 206). In other words, the patient 102 opts to create the first patient profile with the healthcare server 110. In another scenario, the patient 102 may be an existing user of the service application 114 and may select the first log-in option for logging into the patient profile that already exists.

On selection of the first sign-up option, the service application 114 may prompt the patient 102 to provide patient details required for creating the first patient profile (as shown by arrow 208). The patient details required for creating the first patient profile may include a username and a password. The patient details may further include personal details (e.g., a name, an age, a gender, contact details, or the like) of the patient 102 and medical details (e.g., the name of the insurance provider, the insurance policy identifier of the insurance policy, the policy coverage amount of the insurance policy, the medical history of the patient 102, or the like) of the patient 102. The patient 102 submits the requested patient details for creating the first patient profile (as shown by arrow 210). The patient device 104 communicates the submitted patient details to the healthcare server 110 (as shown by arrow 212).

Based on the received patient details, the healthcare server 110 may create the first patient profile and store the first patient profile in the memory of the healthcare server 110 (as shown by arrow 214). The healthcare server 110 may communicate a first notification to the patient device 104, indicating a successful creation of the first patient profile (as shown by arrow 216). Based on the first notification, the patient device 104 displays a first message on the first UI indicating that the first patient profile is successfully created. Thus, the patient 102 is successfully registered with the healthcare server 110 for availing the medical care service. In another embodiment, the patient 102 may choose not to create the first patient profile and may avail the medical care service offered by the healthcare server 110 as a guest user.

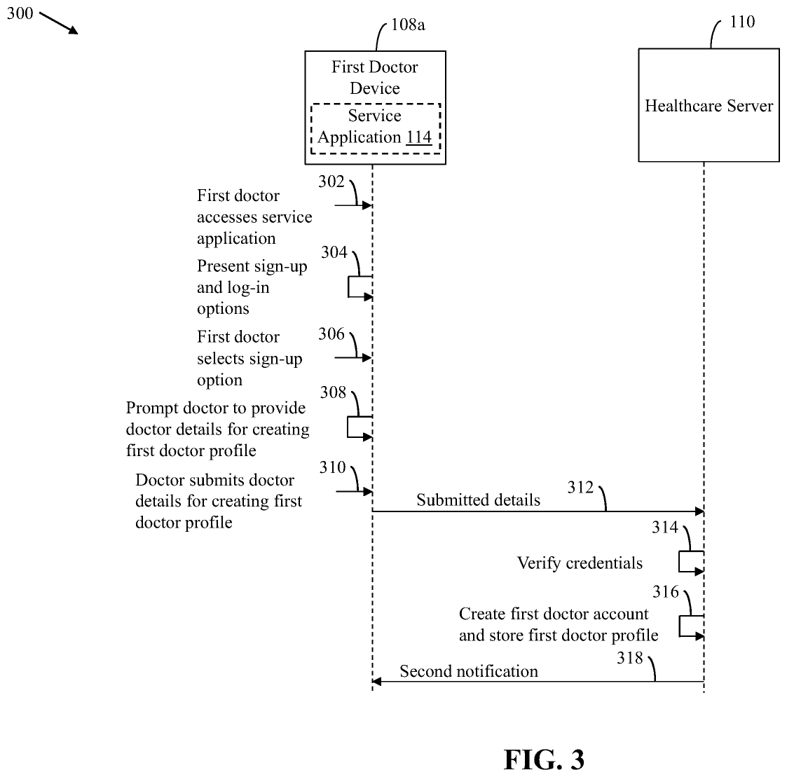

FIG. 3 represents a process flow diagram 300 that illustrates an exemplary scenario for creating the first doctor profile of the first doctor 106a with the healthcare server 110, in accordance with an embodiment of the present invention. The process flow diagram 300 is explained with reference to creation of the first doctor profile of the first doctor 106a. It will be apparent to those of skill in the art that the second and third doctor profiles of the second and third doctors 106b and 106c may be created in a similar manner. The process flow diagram 300 involves the first doctor device 108a and the healthcare server 110.

The first doctor device 108a may be utilized by the first doctor 106a to access the service application 114 that runs on the first doctor device 108a (as shown by arrow 302). The service application 114 may render a second user interface (UI) on a display of the first doctor device 108a. The second UI may present second sign-up and second log-in options to the first doctor 106a (as shown by arrow 304). The second sign-up option may allow the first doctor 106a to create a new doctor profile and the second log-in option may allow the first doctor 106a to log into an existing doctor profile. In a non-limiting example, it is assumed that the first doctor 106a is a new user. Thus, the first doctor 106a selects the second sign-up option (as shown by arrow 306). In other words, the first doctor 106a opts to create the first doctor profile with the healthcare server 110. In another scenario, the first doctor 106a may be an existing user of the service application 114 and may select the second log-in option for logging into the first doctor profile that already exists.

On selection of the second sign-up option, the service application 114 may prompt the first doctor 106a to provide doctor details required for creating the first doctor profile (as shown by arrow 308). The doctor details required for creating the first doctor profile may include a username and a password. The doctor details may further include credential proofs of the first doctor 106a, for example, one or more documents that support the credentials of the first doctor 106a. The credential proofs may include, but are not limited to, a proof of the first set of medical qualifications of the first doctor 106a, a proof of the timespan of the medical experience of the first doctor 106a, a proof for a social security number, a photo identity proof, or the like. In one embodiment, the documents may be uploaded by the first doctor 106a way of the service application 114 that runs on the first doctor device 108a.

The first doctor 106a submits the requested doctor details for creating the first doctor profile (as shown by arrow 310). The first doctor device 108a communicates the submitted doctor details to the healthcare server 110 (as shown by arrow 312). On receiving the doctor details submitted by the first doctor 106a, the healthcare server 110 (or the medical entity associated with the healthcare server 110) may verify the credentials of the first doctor 106a by various methods known in the art (as shown by arrow 314). The various methods may include screening of the first doctor 106a, background checks, or the like. The verification of the credentials of the first doctor 106a may involve manual verification and/or computerized (i.e., automatic) verification. Manual verification may involve contacting associates, acquaintances, clinics and/or hospitals associated with the first doctor 106a for validating the credentials of the first doctor 106a. For automatically verifying the credentials of the first doctor 106a, the healthcare server 110 may refer to a record database and may validate the documents submitted by the first doctor 106a. If the documents submitted by the first doctor 106a are valid and supports the credentials of the first doctor 106a, the healthcare server 110 creates the first doctor profile, else the healthcare server 110 declines the request of the first doctor 106a for creating the first doctor profile. In a non-limiting example, it is assumed that the healthcare server 110 successfully verifies the credentials of the first doctor 106a. Based on the successful verification of the credentials of the first doctor 106a, the healthcare server 110 creates the first doctor profile and stores the first doctor profile in the memory of the healthcare server 110 (as shown by arrow 316). The creation of the first doctor profile indicates that the first doctor 106a is now registered with the healthcare server 110 for delivering medical care to patients. In other words, the first doctor 106a is successfully registered with the healthcare server 110 for delivering medical care. Based on the creation of the first doctor profile, the healthcare server 110 may communicate a second notification to the first doctor device 108a (as shown by arrow 318). The second notification is indicative of the successful registration of the first doctor 106a. Based on the second notification, the service application 114 may display a second message indicating the successful registration of the first doctor 106a.

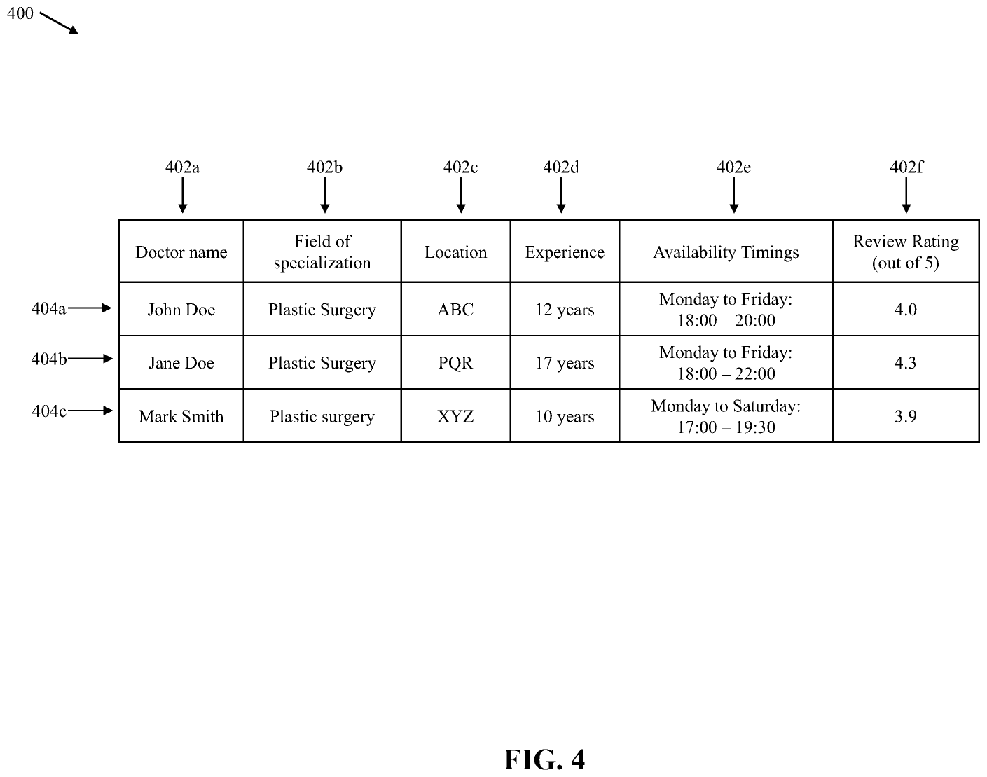

FIG. 4 is a Table 400 that illustrates doctor profiles stored in the memory of the healthcare server 110, in accordance with an embodiment of the invention. Table 400 includes columns 402a-402f and rows 404a-404c. The columns 402a-402f respectively indicate a name, a field of specialization, a location, a timespan of medical experience (i.e., work experience), availability timings, and a review rating of a corresponding doctor 106. The location of a registered doctor included in Table 400 may be a location of a hospital or a clinic where the corresponding doctor 106 practices. The availability timings of a registered doctor included in Table 400 may be indicative of timings when the registered doctor is available for accepting patients requests to provide medical care. The review rating of a registered doctor included in Table 400 may be indicative a cumulative rating provided by various patients to the corresponding doctor based on medical care delivered by the corresponding doctor to the patients.

The row 404a indicates that the first doctor 106a (i.e., “John Doe”) is a specialist in plastic surgery and has “12” years of medical experience. The row 404a further indicates that the first doctor 106a is located at a location “ABC”, is available from Monday to Friday between 18:00 hours (i.e., 6:00 p.m.) and 20:00 hours (i.e., 8:00 p.m.), and has a review rating of “4.0” out of “5”. The row 404b indicates that the second doctor 106b (i.e., “Jane Doe”) is a specialist in plastic surgery and has “17” years of medical experience. The row 404b further indicates that the second doctor 106b is located at a location “PQR”, is available from Monday to Friday between 18:00 hours (i.e., 6:00 p.m.) and 22:00 hours (i.e., 10:00 p.m.), and has a review rating of “4.3” out of “5”. The row 404c indicates that the third doctor 106c (i.e., “Mark Smith”) is a specialist in plastic surgery and has “10” years of medical experience. The row 404c further indicates that the third doctor 106c is available from Monday to Saturday between 17:00 hours (i.e., 5:00 p.m.) and 19:30 hours (i.e., 7:30 p.m.), is located at a location “XYZ”, and has a review rating of “3.9” out of “5”. The doctors 106 may utilize the corresponding doctor devices 108 to update the corresponding availability timings. For example, the first doctor 106a may utilize the service application 114 that runs on the first doctor device 108a to update the availability timings.

Information indicated by Table 400 is merely exemplary and should not be construed to limit the scope of the invention. It will be apparent to those of skill in the art that Table 400 may also include other pertinent information (e.g., educational qualifications of the registered doctors 106, allegations, if any, of malpractice against the doctors 106, or the like) without deviating from the scope of the invention.

FIGS. 5A and 5B, collectively represent a process flow diagram 500 that illustrates a method for delivering medical care to the patient 102, in accordance with an embodiment of the present invention. For the sake of ongoing description of FIGS. 5A and 5B, it is assumed that the patient 102 uses the service application 114 that runs on the patient device 104 for requesting medical care. The process flow diagram 500 involves the patient device 104, the first doctor device 108a, and the healthcare server 110. In a non-limiting example, it is assumed that the patient 102 has met with an accident and is in requirement of a first medical procedure (e.g., plastic surgery) for treating injuries caused due to the accident.

The patient 102 or another user may access the patient device 104 or a user device of the user, respectively, to request medical care for the patient 102. In a non-limiting example, it is assumed that the patient 102 accesses the service application 114 that runs on the patient device 104 to request medical care (as show by arrow 502). In other words, the patient 102 accesses the service application 114 for requesting a doctor for performing the first medical procedure (i.e., plastic surgery). When the patient 102 accesses the service application 114, the service application 114 renders the first UI on the display of the patient device 104. In one embodiment, the patient 102 may access the service application 114 using the username and password associated with the first patient profile of the patient 102. In another embodiment, the patient 102 may access the service application 114 as a guest user. When the patient 102 accesses the service application 114 (i.e., the patient 102 performs the first activity on the patient device 104), the patient device 104 communicates an access notification to the healthcare server 110 (as shown by arrow 504). Based on the access notification, the healthcare server 110 determines a location of the patient 102 (as shown in arrow 506). In one embodiment, the healthcare server 110 may determine the location of the patient 102 by identifying an internet protocol (IP) address of the patient device 104. The IP address may be included in the access notification received by the healthcare server 110. In another embodiment, the healthcare server 110 may request the patient device 104 to communicate location details of the location of the patient device 104 to the healthcare server 110. Based on the request from the healthcare server 110, the patient device 104 may determine a real-time location of the patient device 104 and communicate the location details to the healthcare server 110. The patient device 104 may determine the real-time location of the patient device 104 by using a global positioning system (GPS) of the patient device 104. In another embodiment, the healthcare server 110 may prompt the patient 102, by way of the service application 114, to provide the location details of the current location of the patient 102. The patient 102 may then submit the location details to the patient device 104. The patient device 104 then communicates the location details submitted by the patient 102 to the healthcare server 110. The healthcare server 110 may further prompt the patient 102 to provide details pertaining to the injury that needs to be treated. For example, the healthcare server 110 may prompt the patient 102 to provide details pertaining to the injury such as, but not limited to, a cause of the injury, a location (e.g., face) of the injury, or the like. The healthcare server 110 may further prompt the patient 102 to upload photographs of the injury.

Based on the location of the patient 102 and details of the injury, the healthcare server 110 may identify a first set of doctors for providing medical care to the patient 102 (as shown by arrow 508). The healthcare server 110 may use Table 400, the location of the patient 102, and the details of the injury as filtering parameters for identifying the first set of doctors. For example, the healthcare server 110 may include, in the first set of doctors, only those doctors that are located within a pre-determined distance (e.g., “5 kilometer, km”) from the location of the patient 102 and are capable of treating the injury of the patient 102. The pre-determined distance may be set by the healthcare server 110 or by the patient 102. In a non-limiting example, it is assumed that the locations of the doctors 106 as specified in Table 400 are within the pre-determined distance of the location of the patient 102 and the doctors 106 are capable of performing the first medical procedure to treat the injury of the patient 102. The healthcare server 110 further determines that the available timings of the doctors 106 as specified in Table 400 cover the time instance at which the patient 102 requires medical care. Thus, the healthcare server 110 includes the doctors 106 in the first set of doctors.

Based on the identification of the first set of doctors, the healthcare server 110 presents first and second options to the patient for selection. The healthcare server 110 may present the first and second options by way of the first UI of the service application 114 (as shown by arrow 510). The first option, when selected by the patient 102, enables the patient 102 to choose one or more doctors from the first set of doctors and the second option, when selected by the patient 102, causes the healthcare server 110 to automatically select a suitable doctor for the patient 102. For the sake of ongoing description of FIGS. 5A and 5B, it is assumed that the patient 102 selects the first option (as shown by arrow 512). An exemplary scenario in which the patient 102 selects the second option is explained in detail in conjunction with FIGS. 6A and 6B.

The patient device 104 may communicate a first selection notification to the healthcare server 110 to indicate that the patient 102 has selected the first option (as shown by arrow 514). Based on the first selection notification, the healthcare server 110 communicates information pertaining to the first set of doctors (i.e., the doctors 106) to the patient device 104 (as shown by arrow 516). The information pertaining to the first set of doctors may include the first through third doctor profiles of the doctors 106, respectively. The patient 102 may assess the doctors 106 by viewing the first through third doctor profiles. Based on the assessment, the patient 102 may choose a subset of doctors from the first set of doctors (as shown by arrow 518). In one example, the patient 102 may choose only one doctor from the first set of doctors. For example, the patient 102 may choose a doctor in the first set of doctors who has the highest review rating. In another example, the patient 102 may choose the most experienced doctor in the first set of doctors. In another example, the patient 102 may choose more than one doctor from the first set of doctors. For example, the patient 102 may choose two doctors in the first set of doctors who have the highest review ratings. In a non-limiting example, it is assumed that, among the first through third doctors 106a-106c, the patient 102 selects the first doctor 106a to perform the first medical procedure for treating the injury of the patient 102. Based on the selection by the patient 102, the patient device 104 communicates a second selection notification to the healthcare server 110 (as shown by arrow 520). The second selection notification is indicative of the subset of doctors (e.g., the first doctor 106a) selected by the patient 102.

With reference to FIG. 5B, based on the second selection notification, the healthcare server 110 communicates a first availability request to the first doctor device 108a of the selected first doctor 106a (as shown by arrow 522). The first availability request is a request for the first doctor 106a to confirm an availability (i.e., an availability confirmation) for performing the first medical procedure. The first availability request may be in the form of a short message service (SMS) text, an email, or an in-app notification communicated by way of the service application 114. The first availability request may include various response codes that the first doctor 106a may use while responding to the first availability request. For example, a first response code in the first availability request may indicate that the first doctor 106a is available for performing the first medical procedure. Likewise, a second response code in the first availability request may indicate that the first doctor 106a is unavailable for performing the first medical procedure. The first availability request further includes a validity information. The validity information is indicative of a time-period (e.g., “5” minutes) within which the first doctor 106a has to reply to the first availability request. The first doctor 106a may enter an appropriate response code and initiate a first availability response by way of the first doctor device 108a. The first doctor device 108a communicates the first availability response to the healthcare server 110 (as shown by arrow 524). In a non-limiting example, it is assumed that the first availability response includes the first response code, i.e., the first doctor 106a is available for performing the first medical procedure. When the first availability response is received by the healthcare server 110, the healthcare server 110 determines a response time of the first availability response (as shown by arrow 526). The response time is indicative of a time duration taken by the first doctor 106a to respond to the first availability request. For example, the response time of the first availability response is the difference between a first timestamp at which the first availability request is communicated to the first doctor device 108a and a second timestamp at which the first availability response is received from the first doctor device 108a. If the response time is greater than the time-period indicated by the validity information, the healthcare server 110 discards the first availability response and notifies the patient 102 that the first doctor 106a is unavailable. In such a scenario, the healthcare server 110 may provide automatic recommendation of one or more alternate doctors to the patient 102. In a non-limiting example, it is assumed that the first availability response is received before the expiry of the time-period indicated by the validity information.

Based on the first availability response, the healthcare server 110 may select the first doctor 106a for performing the first medical procedure on the patient 102 (as shown by arrow 528). Based on the selection of the first doctor 106a, the healthcare server 110 may communicate a third notification to the patient device 104, apprising the patient 102 of the selection of the first doctor 106a for performing the first medical procedure (as shown by arrow 530). Consequently, the healthcare server 110 may communicate the first patient profile including the patient details of the patient 102 to the first doctor device 108a, requesting the first doctor 106a to contact the patient 102 (as shown by arrow 532). Based on the contact details included in the first patient profile, the first doctor 106a may communicate with the patient 102 and arrange a meeting with the patient 102 for assessing the patient 102 and performing the first medical procedure. In another embodiment, by way of the service application 114, the healthcare server 110 may establish a real-time electronic communication link between the patient device 104 and the first doctor device 108a of the first doctor 106a. By way of the communication link, the patient 102 and the first doctor 106a may communicate with each other and arrange the meeting.

Upon completion of the first medical procedure, the first doctor 106a may upload documents (e.g., prescriptions, notes regarding post-operative care, or the like) related to the first medical procedure to the healthcare server 110, by way of the service application 114. The documents may be in the form of text, images, or the like. The first doctor 106a may further upload insurance information of the patient 102 and patient-specific information such as demographics of the patient 102, or the like. The patient 102 may write a review of the first medical procedure and/or rate the first doctor 106a, by way of the service application 114. The healthcare server 110 may further update the review rating of the first doctor 106a based on the rating provided by the patient 102.

In another embodiment, the healthcare server 110 may prompt the patient 102, by way of the service application 114, to provide one or more doctor preferences that the patient 102 has for selecting a suitable doctor. The doctor preferences provided by the patient 102 may include a type of doctor required, a timespan of medical experience of the doctor, a review rating threshold of the doctor, or the like. The healthcare server 110 may utilize the doctor preferences provided by the patient 102 as additional filtering parameters for identifying the first set of doctors. In another embodiment, the healthcare server 110 allows the patient 102 to request for immediate medical care or schedule an appointment with a doctor. In a scenario, where the patient 102 schedules an appointment with a doctor, the patient 102 may include a preferred time, date, and location for meeting with the doctor for the appointment.

FIGS. 6A and 6B, collectively represent a process flow diagram 600 that illustrates a method for delivering medical care to the patient 102, in accordance with another embodiment of the present invention. For the sake of ongoing description of FIGS. 6A and 6B, it is assumed that the patient 102 uses the service application 114 that runs on the patient device 104 for requesting medical care. The process flow diagram 600 involves the patient device 104, the first and second doctor devices 108a and 108b, and the healthcare server 110. FIGS. 6A and 6B have been explained in conjunction with FIG. 5A.

The patient device 104 receives the first message and presents the first and second options on the first UI (as shown by arrow 510). In a non-imitating example, it is assumed that the patient 102 selects the second option (as shown by arrow 602). The patient device 104 may communicate a third selection notification to the healthcare server 110 to indicate that the patient 102 has selected the second option (as shown by arrow 604). Based on the third selection notification, the healthcare server 110 automatically selects a subset of doctors from the first set of doctors (as shown by arrow 606). The subset of doctors may include those doctors that satisfy the doctor preferences provided by the patient 102. In a scenario, where the patient 102 has not provided any doctor preferences, the subset of doctors may be same as the first set of doctors. In a non-limiting example, it is assumed that the subset of doctors identified by the healthcare server 110 include the first and second doctors 106a and 106b.

Based on the identification of the subset of doctors, the healthcare server 110 may communicate the first availability request to the first and second doctor devices 108a and 108b (as shown by arrows 608a and 608b). The healthcare server 110 may receive a second availability response and a third availability response from the first doctor device 108a and the second doctor device 108b, respectively (as shown by arrows 610a and 610b). In a non-limiting example, it is assumed that both the second and third availability responses are indicative of the first response code, i.e., the first and second doctors 106a and 106b are available for performing the first medical procedure on the patient 102.

With reference to FIG. 6B, after receiving the second and third availability responses, the healthcare server 110 determines response times of the second and third availability responses (as shown by arrow 612). In a non-limiting example, it is assumed the response time of the second availability response is less than the response time of the third availability response. In other words, the second availability response was communicated to the healthcare server 110 before the third availability response. Therefore, based on the response time of the second availability response, the healthcare server 110 may select the first doctor 106a for performing the first medical procedure on the patient 102 (as shown by arrow 614). Based on the selection of the first doctor 106a, the healthcare server 110 may communicate a fourth notification to the patient device 104, apprising the patient 102 of the selection of the first doctor 106a for performing the first medical procedure (as shown by arrow 616). Consequently, the healthcare server 110 may communicate the first patient profile including patient details of the patient 102 to the first doctor device 108a, requesting the first doctor 106a to contact the patient 102 (as shown by arrow 618). Based on the contact details included in the first patient profile, the first doctor 106a may communicate with the patient 102 and arrange a meeting with the patient 102 for assessing the patient 102 and performing the first medical procedure. In another embodiment, by way of the service application 114, the healthcare server 110 may establish a real-time electronic communication link between the patient device 104 and the first doctor device 108a of the first doctor 106a. By way of the communication link, the patient 102 and the first doctor 106a may communicate with each other and arrange the meeting.

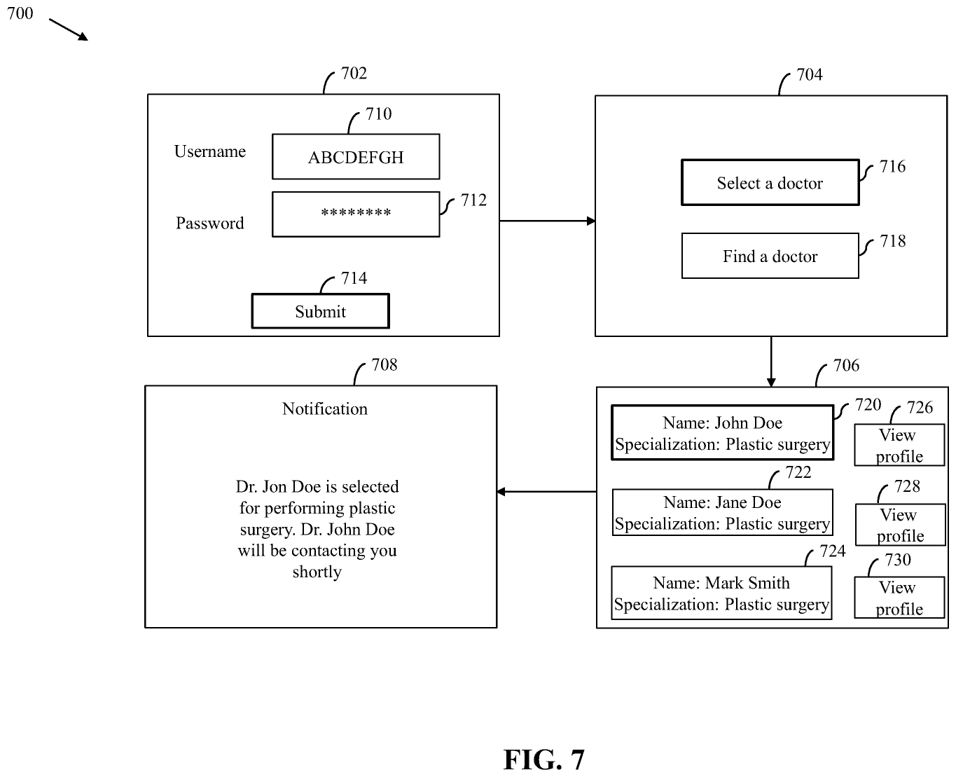

FIG. 7 represents an exemplary scenario 700 that illustrates first through fourth UI screens 702, 704, 706, and 708 rendered by the service application 114, in accordance with an embodiment of the present invention. The UI screens 702-708 are rendered on the display (not shown) of the patient device 104. FIG. 7 has been explained in conjunction with FIGS. 5A and 5B.

When the patient 102 accesses the service application 114, the service application 114 renders the UI screen 702 on the display of the patient device 104. The UI screen 702 prompts the patient 102 to enter the username and password associated with the first patient profile to log into the service application 114. The UI screen 702 may include first and second text boxes 710 and 712 for entering the username and password associated with the first patient profile, respectively. The UI screen 702 further includes a first submit button 714. The service application 114 serves as a gateway to the healthcare server 110, and thus, when the patient 102 selects the first submit button 714, an authentication request that includes the username and password associated with the first patient profile is communicated to the healthcare server 110 for authentication of the patient 102. Based on the username and password, the healthcare server 110 may communicate an authentication response to the patient device 104. The authentication response may be indicative of a successful or a failed authentication. In a non-limiting example, the authentication response is indicative of a successful authentication. Based on the authentication response, the service application 114 renders the UI screen 704.

The UI screen 704 may include the first and second options 716 and 718 that are user-selectable. The first option 716 allows the patient 102 to select a doctor for performing the first medical procedure. The second option 718 allows the healthcare server 110 to automatically select a suitable doctor for performing the first medical procedure. In another embodiment, the UI screen 704 may further include options for requesting an immediate appointment or scheduling an appointment with a doctor. In a non-limiting example, it is assumed that the patient 102 is requesting for an immediate appointment (i.e., immediate medical care). In a non-limiting example, the patient 102 may select the first option 716. Based on the selection of the first option 716, the patient device 104 may communicate the first selection notification to the healthcare server 110. Based on the first selection notification, the healthcare server 110 may communicate the information pertaining to the first set of doctors to the patient device 104. Based on the information, the service application 114 may render the UI screen 706. In another embodiment, if the patient 102 selects the second user-selectable option 718, the service application 114 may render a UI screen, prompting the patient 102 for doctor preferences of the patient 102 and directly renders the UI screen 708 on the patient device 104 to present the selected doctor to the patient 102.

The UI screen 706 may include third through fifth user-selectable options 720, 722, and 724 that allow the patient 102 to select the first through third doctors 106a-106c, respectively. The UI screen 706 may further include sixth through eighth user-selectable options 726, 728, and 730 for viewing the first through third doctor profiles, respectively. The patient 102 may select the sixth through eighth user-selectable options 726, 728, and 730 for viewing the first through third doctor profiles, respectively. The patient 102 may select one or more of the third through fifth user-selectable options 720, 722, and 724. In one embodiment, when the patient 102 selects one or more of the sixth through eighth user-selectable options 726, 728, and 730, the healthcare server 110 prompts the patent to provide one or more details pertaining to the injury. Further, in another embodiment, when the patient 102 accesses the service application 114 as a guest user, the healthcare server 110 prompts the patient 102 to provide personal and medical details.

In a non-limiting example, the patient 102 selects the third user-selectable option 720 (i.e., the first doctor 106a). Based on the selection, the patient device 104 may communicate the second selection notification to the healthcare server 110. The healthcare server 110 communicates the first availability request to doctor devices (i.e., the first doctor device 108a) of a subset of doctors (i.e., the first doctor 106a selected by the patient 102). Based on the first availability response from the first doctor 106a, the healthcare server 110 communicates the third notification to the patient device 104. Based on the third notification, the service application 114 renders the UI screen 708. The UI screen 708 includes a message that indicates that the first doctor 106a (i.e., Dr. John Doe) is selected for performing the first medical procedure and that the first doctor 106a will be contacting the patient 108 shortly.

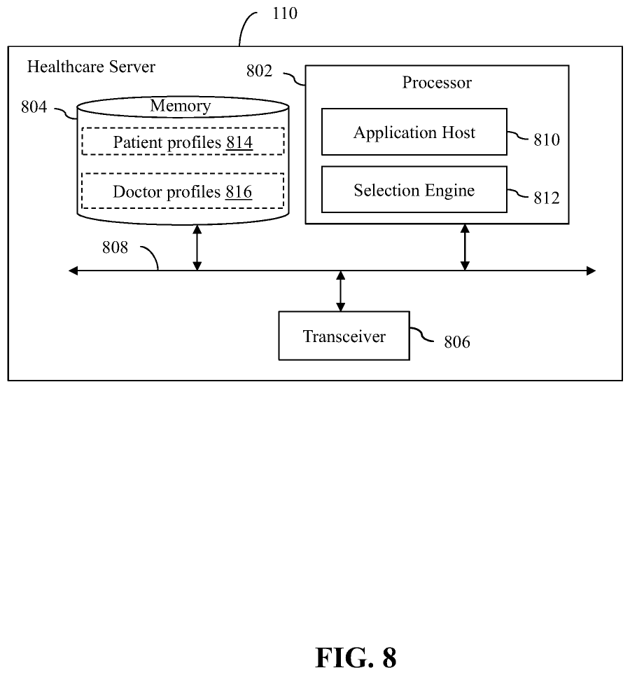

FIG. 8 is a block diagram that illustrates the healthcare server 110, in accordance with an embodiment of the present invention. The healthcare server 110 includes a processor 802, a memory 804, and a transceiver 806. The processor 802, the memory 804, and the transceiver 806 communicate with each other by way of a communication bus 808. The processor 802 includes an application host 810 and a selection engine 812.

The processor 802 may include suitable logic, circuitry, interfaces, and/or codes, executed by the circuitry, that may be configured to execute one or more operations for delivering medical care to the patients (e.g., the patient 102). The processor 802 may store, in the memory 804, patient profiles 814 of patients who have signed up for availing the medical care service and doctor profiles 816 of the doctors have registered with the healthcare server 110 for delivering medical care to the patients. The processor 802 hosts the service application 114 that is executable on the patient device 104 and the doctor devices 108. The processor 802 may authenticate the patient 102 when the patient 102 attempts to log into the service application 114.

Examples of the processor 802 may include, but are not limited to, an application-specific integrated circuit (ASIC) processor, a reduced instruction set computer (RISC) processor, a complex instruction set computer (CISC) processor, a field programmable gate array (FPGA), and the like. The processor 802 executes operations for selecting doctors for performing medical procedures (e.g., the first medical procedure) on the patients by way of the application host 810 and the selection engine 812.

The application host 810 executes operations for hosting the service application 114 that is executable on various devices, such as the patient device 104 and the doctor devices 108. The application host 810 may control the service application 114 and cause it to perform various operations (such as the rendering of the UI screens 702-708) as described in FIGS. 7A and 7B. By way of the UI screens 702-708, the application host 810 allows the patients (e.g., the patient 102) to avail the medical care service. By way of the UI screens 702-708, the application host 810 allows patients to initiate requests (e.g., the first request) for medical care. The application host 810 creates and stores the patient profiles 814 of the patients and the doctor profiles 816 of the doctors 106 in the memory 804.

The selection engine 812 facilitates selection of doctors (such as the first doctor 106a) for delivering medical care to the patients, based on requests (such as the first request) initiated by the patients. The selection engine 812 determines locations of patient devices (e.g., the patient device 104) of the patients (e.g., the patient 102) and determines sets of doctors (such as the first set of doctors) based on the locations of the patient devices and doctor preferences of the patients. The selection engine 812 may communicate with doctors (such as the first and second doctors 106a and 106b) for confirming availabilities of the doctors and select doctors for performing medical procedures based on response times of availability responses of the doctors to the availability requests as described in the foregoing description of FIGS. 2-7.

The memory 804 includes suitable logic, circuitry, interfaces, and/or codes, executable by the circuitry, to store the patient profiles 814 of the patients and the doctor profiles 816 of the doctors. Examples of the memory 804 may include a random-access memory (RAM), a read-only memory (ROM), a removable storage drive, a hard disk drive (HDD), a flash memory, a solid-state memory, and the like. It will be apparent to a person skilled in the art that the scope of the invention is not limited to realizing the memory 804 in the healthcare server 110, as described herein. In another embodiment, the memory 804 may be realized in form of a database server or a cloud storage working in conjunction with the healthcare server 110, without departing from the scope of the invention.

The transceiver 806 includes suitable logic, circuitry, interfaces, and/or codes, executable by the circuitry, for transmitting and receiving data over the communication network 112 using one or more communication network protocols. The transceiver 806 transmits various requests and messages to the patient device 104 and the doctor devices 108. The transceiver 806 further receives various requests and messages from the patient device 104 and the doctor devices 108. Examples of the transceiver 806 may include, but are not limited to, an antenna, a radio frequency transceiver, a wireless transceiver, a Bluetooth transceiver, an ethernet port, a universal serial bus (USB) port, or any other device configured to transmit and receive data.

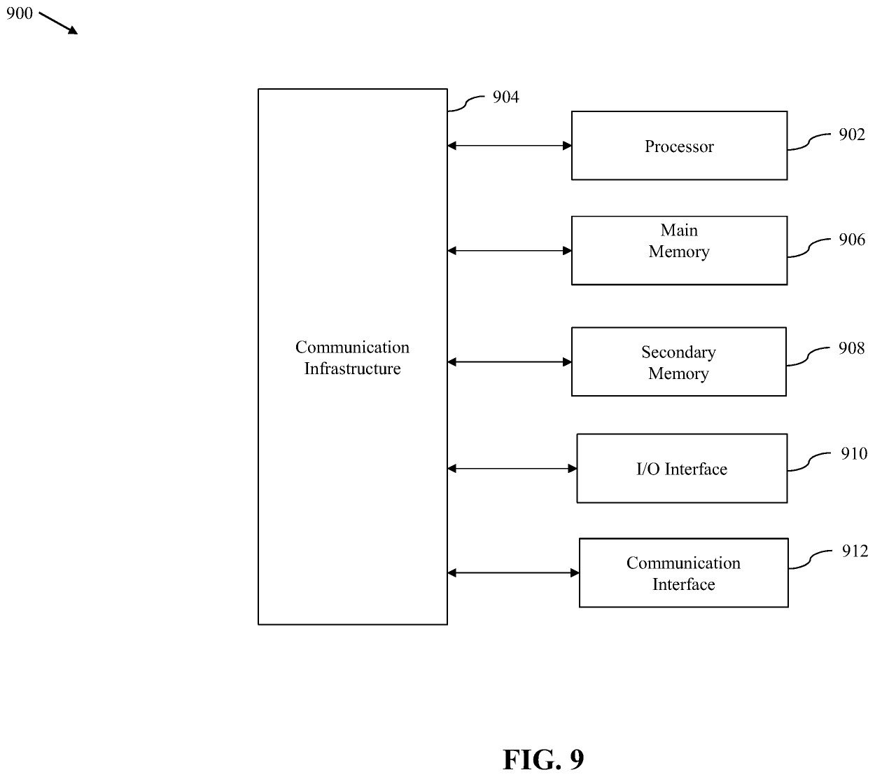

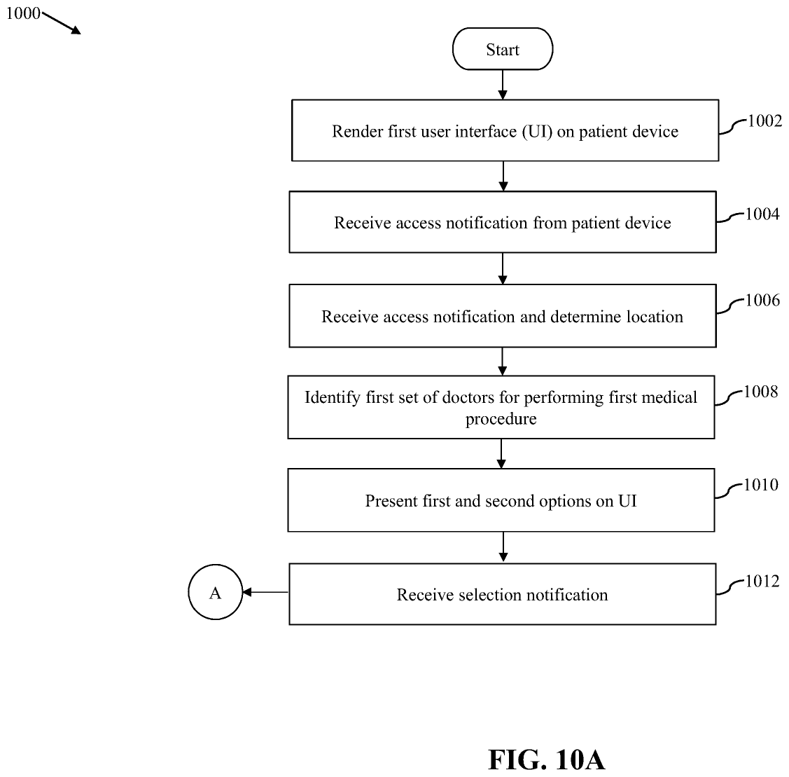

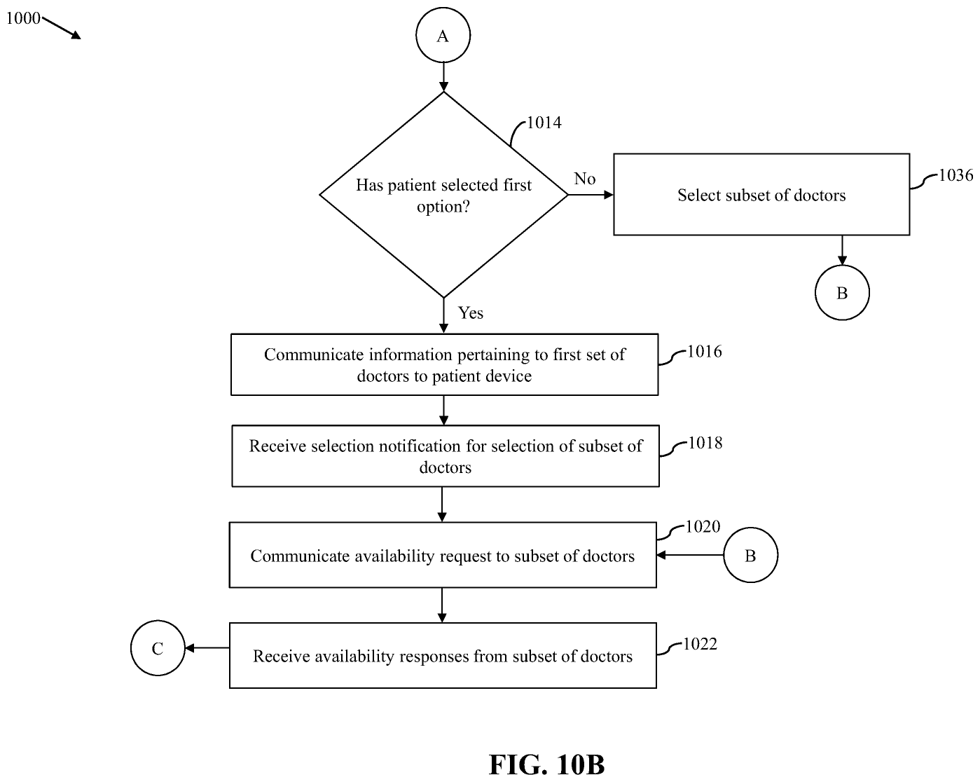

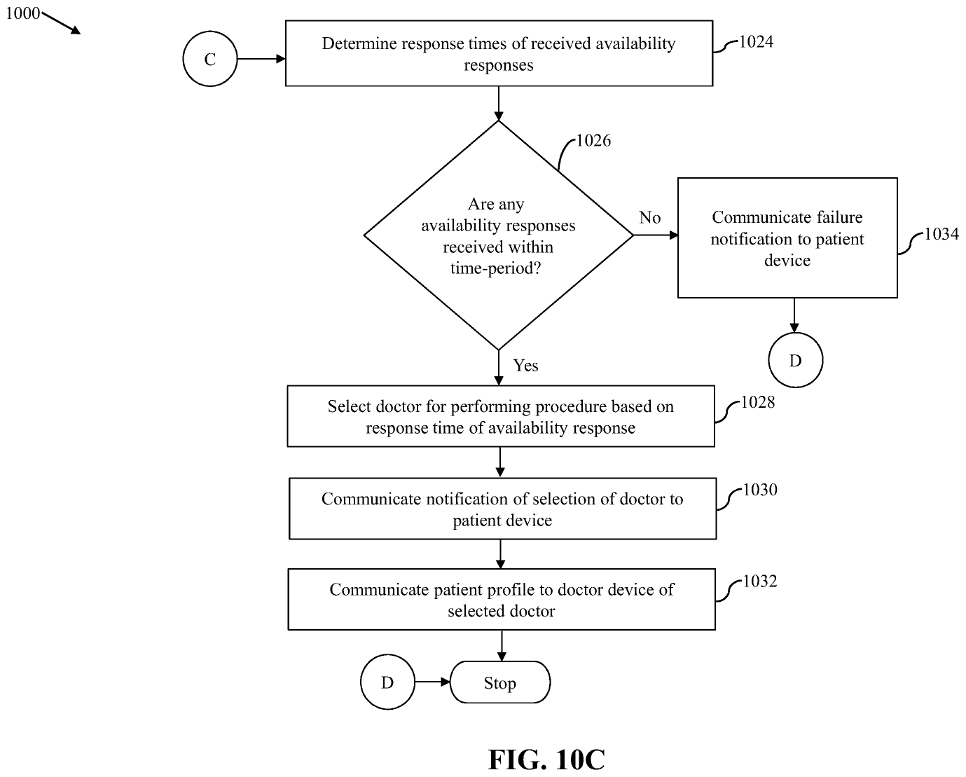

FIG. 9 is a block diagram that illustrates system architecture of a computer system 900, in accordance with an embodiment of the present invention. An embodiment of present invention, or portions thereof, may be implemented as computer readable code on the computer system 900. In one example, the patient device 104, the doctor devices 108, and the healthcare server 110 may be implemented in the computer system 900 using hardware, software, firmware, non-transitory computer readable media having instructions stored thereon, or a combination thereof and may be implemented in one or more computer systems or other processing systems. Hardware, software, or any combination thereof may embody modules and components used to implement the methods of FIGS. 10A-10C.

The computer system 900 includes a processor 902 that may be a special-purpose or a general-purpose processing device. The processor 902 may be a single processor, multiple processors, or combinations thereof. The processor 902 may have one or more processor cores. In one example, the processor 902 is an octa-core processor. The processor 902 may be connected to a communication infrastructure 904, such as a bus, message queue, multi-core message-passing scheme, and the like. The computer system 900 may further include a main memory 906 and a secondary memory 908. Examples of the main memory 906 may include RAM, ROM, and the like. The secondary memory 908 may include a hard disk drive or a removable storage drive, such as a floppy disk drive, a magnetic tape drive, a compact disc, an optical disk drive, a flash memory, and the like. The removable storage drive may read from and/or write to a removable storage device in a manner known in the art. In one example, if the removable storage drive is a compact disc drive, the removable storage device may be a compact disc. In an embodiment, the removable storage unit may be a non-transitory computer readable recording media.

The computer system 900 further includes an input/output (I/O) interface 910 and a communication interface 912. The I/O interface 910 includes various input and output devices that are configured to communicate with the processor 902. Examples of the input devices may include a keyboard, a mouse, a joystick, a touchscreen, a microphone, and the like. Examples of the output devices may include a display screen, a speaker, headphones, and the like. The communication interface 912 may be configured to allow data to be transferred between the computer system 900 and various devices that are communicatively coupled to the computer system 900. Examples of the communication interface 912 may include a modem, a network interface, i.e., an Ethernet card, a communication port, and the like. Data transferred via the communication interface 912 may correspond to signals, such as electronic, electromagnetic, optical, or other signals as will be apparent to a person skilled in the art. The signals may travel via a communication channel (not shown) which may be configured to transmit the signals to devices that are communicatively coupled to the computer system 900. Examples of the communication channel may include, but are not limited to, cable, fiber optics, a phone line, a cellular phone link, a radio frequency link, and the like.

The main memory 906 and the secondary memory 908 may refer to non-transitory computer readable mediums. These non-transitory computer readable mediums may provide data that enables the computer system 900 to implement the methods illustrated in FIGS. 10A-10C. In an embodiment, the present invention is implemented using a computer implemented application, the computer implemented application may be stored in the main memory 906 and/or the secondary memory 908.

A person having ordinary skill in the art will appreciate that embodiments of the disclosed subject matter can be practiced with various computer system configurations, including multi-core multiprocessor systems, minicomputers, mainframe computers, computers linked or clustered with distributed functions, as well as pervasive or miniature computers that may be embedded digitally into any device. For instance, at least one processor such as the processor 902 and a memory such as the main memory 906 and the secondary memory 908 implements the above described embodiments. Further, the operations may be described as a sequential process, however some of the operations may in fact be performed in parallel, concurrently, and/or in a distributed environment, and with program code stored locally or remotely for access by single or multiprocessor machines. In addition, in some embodiments the order of operations may be rearranged without departing from the spirit of the disclosed subject matter.

FIGS. 10A-10C, collectively represent a flow chart 1000 that illustrates a method for delivering medical care to the patient 102, in accordance with an embodiment of the present invention. The patient 102 accesses the service application 114 that runs on the patient device 104.

At step 1002, the healthcare server 110 renders the first UI when the patient 102 accesses the service application 114. The healthcare server 110 may prompt the patient 102 to enter the username and password, associated with the first patient profile, to log into the service application 114. The patient 102 may enter the username and password for logging into the service application 114 or may access the service application 114 as a guest user. At step 1004, the healthcare server 110 receives the access notification from the patient device 104. At step 1006, the healthcare server 110 determines the location of the patient device 104 based on the access notification. At step 1008, the healthcare server 110 identifies the first set of doctors for performing the first medical procedure based on the determined location. At step 1010, the healthcare server 110 presents the first and second options 716 and 718 on the first UI on the patient device 104. At step 1012, the healthcare server 110 receives a selection notification (e.g., the first selection notification) from the patient device 104. The selection notification may indicate whether the patient 102 has selected the first option 716 or the second option 718.

With reference to FIG. 10B, at step 1014, the healthcare server 110 determines, based on the selection notification, whether the patient 102 has selected the first option 716. If at step 1014, it is determined that the patient 102 has selected the first option, step 1016 is performed. At step 1016, the healthcare server 110 communicates the information pertaining to the first set of doctors to the patient device 104. The service application 114 may display the first through third doctor profiles of the doctors 106 (i.e., the first set of doctors). At step 1018, the healthcare server 110 receives another selection notification (e.g., the second selection notification) from the patient device 104 indicating a subset of doctors selected by the patient 102 from the first set of doctors. At step 1020, the healthcare server 110 communicates availability requests (e.g., the first availability request) requests to doctor devices of the subset of doctors. At step 1022, the healthcare server 110 receives availability responses (e.g., the first availability response) from doctor devices (e.g., the first doctor device 108a) of the subset of doctors.

With reference to FIG. 10C, at step 1024, the healthcare server 110 determines response times of the received availability responses (e.g., the first availability response). At step 1026, the healthcare server 110 determines whether any availability response, of the received availability responses, is received within the time-period indicated by the validity information of the availability request. If at step 1026, it is determined that one or more availability responses are received within the time-period, step 1028 is performed.

At step 1028, the healthcare server 110 selects, based on the response times of the received availability responses, a doctor (e.g., the first doctor 106a) for performing the first medical procedure. For example, the healthcare server 110 selects that doctor whose response time to the availability request is minimum. At step 1030, the healthcare server 110 communicates a notification to the patient device 104, indicating that the first doctor 106a is selected for performing the first medical procedure. At step 1032, the healthcare server 110 communicates the first patient profile to the first doctor device 108a, requesting the first doctor 106a to contact the patient 102.

If at step 1026, it is determined that no availability response is received within the time-period, step 1034 is performed. At step 1034, the healthcare server 110 communicates a notification to the patient device 104, apprising the patient 102 of a failure to select a doctor for delivering medical care to the patient 102. The healthcare server 110 may select a new subset of doctors for delivering medical care to the patient 102 and repeat steps 1020-1032.

If at step 1014 (as shown in FIG. 10B), it is determined that the patient 102 has selected the second option, step 1036 is performed. At step 1036, the healthcare server 110 automatically selects the subset of doctors (as described in the foregoing description of FIG. 6B) and performs step 1020.

Various embodiments of the invention may be found in a disclosed apparatus (for example, the healthcare server 110) for delivering medical care to the patient 102. In an embodiment, the healthcare server 110 identifies, based on the location of the patient, the first set of doctors for performing the first medical procedure. The healthcare server 110 communicates the first availability request to the subset of the first set of doctors, requesting confirmation of availability of each doctor of the subset of doctors of the first set of doctors for performing the first medical procedure. The healthcare server 110 receives from one or more doctors of the subset of doctors, availability responses (e.g., the first, second, and third availability responses). Each availability response indicates availability of a corresponding doctor 106 for performing the first medical procedure. The healthcare server 110 selects the first doctor 106a from the one or more doctors, for performing the first medical procedure. The first doctor 106a is selected based on a response time (e.g., the first response time) of an availability response (e.g., the first availability response) of the first doctor 106a to the first availability request.

Various embodiments of the invention provide a non-transitory computer readable medium having stored thereon, computer executable instructions, which when executed by a computer, cause the computer to execute operations for delivering medical care to the patients (for example, the patient 102). The operations include identifying, based on the location of the patient, the first set of doctors for performing the first medical procedure. The operations further include communicating the first availability request to the subset of the first set of doctors, requesting confirmation of availability of each doctor of the subset of doctors of the first set of doctors for performing the first medical procedure. The operations further include receiving from one or more doctors of the subset of doctors, availability responses (e.g., the first, second, and third availability responses). Each availability response indicates availability of a corresponding doctor 106 for performing the first medical procedure. The operations further include selecting the first doctor 106a from the one or more doctors, for performing the first medical procedure. The first doctor 106a is selected based on a response time (e.g., the first response time) of an availability response (e.g., the first availability response) of the first doctor 106a to the first availability request.

Thus, the environment 100 facilitates delivery of medical care to patients such as the patient 102. The invention enables patients to request doctors for performing medical procedures. The invention also facilitates quick selection of doctors for providing medical care for patients, in case of emergencies. Using the service application 114 to select doctors for performing the medical procedures, the patients may avoid long waiting periods at medical facilities (e.g., clinics or hospitals), enhancing a quality of medical treatment provided to the patients. Patients may view doctor profiles of a pool of doctors (e.g., the doctors 106) prior to selecting doctors for treatment, allowing the patients to make informed decisions in selecting the doctors. For example, a patient requiring surgery may select a doctor with a high review rating and many years of medical experience. The healthcare server 110 further enables automatic selection of a suitable doctor for performing the medical procedure. As the pool of doctors is filtered based on the injury type, the location, and the preferences of the patient 102, the quality of medical treatment provided to the patient 102 is enhanced and the time for providing medical treatment is reduced. In addition, the patient 102 is saved from the hassle of selecting a suitable doctor at the time of emergencies. The invention is scalable and is capable of catering to any number of patients and doctors.

Techniques consistent with the present invention provide, among other features, systems and methods for delivering medical care to a patient. While various exemplary embodiments of the disclosed system and method have been described above it should be understood that they have been presented for purposes of example only, not limitations. It is not exhaustive and does not limit the invention to the precise form disclosed. Modifications and variations are possible in light of the above teachings or may be acquired from practicing of the invention, without departing from the breadth or scope.

In the claims, the words “comprising”, “including” and “having” do not exclude the presence of other elements or steps then those listed in a claim. The terms “a” or “an,” as used herein, are defined as one or more than one. Unless stated otherwise, terms such as “first” and “second” are used to arbitrarily distinguish between the elements such terms describe. Thus, these terms are not necessarily intended to indicate temporal or other prioritization of such elements. The fact that certain measures are recited in mutually different claims does not indicate that a combination of these measures cannot be used to advantage.

While various embodiments of the present invention have been illustrated and described, it will be clear that the present invention is not limited to these embodiments only. Numerous modifications, changes, variations, substitutions, and equivalents will be apparent to those skilled in the art, without departing from the spirit and scope of the present invention, as described in the claims.

What is claimed is:

1. A method for delivering medical care to a patient, the method comprising:
identifying, by a server, based on a first location of the patient, a first set of doctors for performing a medical procedure on the patient;
communicating, by the server, an availability request to a subset of the first set of doctors, requesting an availability confirmation of each doctor of the subset of doctors for performing the medical procedure;
receiving, by the server from one or more doctor devices, one or more availability responses of one or more doctors, wherein each availability response is indicative of an availability of a corresponding doctor for performing the medical procedure, and wherein the subset of doctors comprises the one or more doctors;
selecting, by the server, a first doctor from the one or more doctors, for performing the medical procedure, wherein the first doctor is selected based on a response time of the availability response of the first doctor to the availability request; and
communicating, by the server to a patient device of the patient, a notification indicating the selection of the first doctor for performing the medical procedure.

2. The method of claim 1, wherein the first set of doctors is located within a pre-determined distance from the first location.

3. The method of claim 1, further comprising:
receiving, by the server, an access notification from the patient device based on a first activity performed by the patient on the patient device; and
determining, by the server, the first location of the patient based on the access notification.

4. The method of claim 1, further comprising:
rendering, by the server, on the patient device, a graphical interface to present first and second options that are selectable by the patient, wherein the first option, when selected by the patient, allows the patient to choose the subset of doctors from the first set of doctors, and wherein the second option, when selected by the patient, allows the server to automatically choose the subset of doctors from the first set of doctors.

5. The method of claim 1, further comprising receiving, by the server from the patient device, a set of preferences provided by the patient, and wherein the availability request is communicated to the subset of doctors such that each doctor of the subset of doctors satisfies the set of preferences.

6. The method of claim 5, wherein the set of preferences includes a type of doctor, a timespan of medical experience of the doctor, or a review rating threshold of the doctor.

7. The method of claim 1, further comprising:
receiving, by the server from a plurality of doctor devices, a plurality of credential proofs of a plurality of doctors;
verifying, by the server, credentials of each doctor of the plurality of doctors based on the plurality of credential proofs; and
registering, by the server, the plurality of doctors based on the verification of the corresponding credentials, wherein the first set of doctors is identified from the registered plurality of doctors.

8. The method of claim 1, further comprising communicating, by the server, patient details of the patient to the first doctor based on the selection of the first doctor, wherein the patient details are indicative of contact information and a medical condition of the patient.

9. The method of claim 1, further comprising rendering, by the server, on the patient device, a graphical interface to present a profile of each of the identified first set of doctors to the patient.

10. The method of claim 9, wherein the profile of each doctor includes at least one of a name of the corresponding doctor, a timespan of medical experience of the corresponding doctor, a specialization of the corresponding doctor, or a review rating of the corresponding doctor.

11. A system for delivering medical care to a patient, the system comprising:
a server configured to:
identify, based on a first location of the patient, a first set of doctors for performing a medical procedure on the patient,
communicate an availability request to a subset of the first set of doctors, requesting an availability confirmation of each doctor of the subset of doctors for performing the medical procedure,
receive, from one or more doctor devices, one or more availability responses of one or more doctors, wherein each availability response is indicative of an availability of a corresponding doctor for performing the medical procedure, and wherein the subset of doctors comprises the one or more doctors,
select a first doctor from the one or more doctors, for performing the medical procedure, wherein the first doctor is selected based on a response time of the availability response of the first doctor to the availability request, and
communicate, to a patient device of the patient, a notification indicating the selection of the first doctor for performing the medical procedure.

12. The system of claim 11, wherein the server is further configured to:
receive an access notification from the patient device based on a first activity performed by the patient on the patient device, and
determine the first location of the patient based on the access notification.

13. The system of claim 11, wherein the server is further configured to render, on the patient device, a graphical interface to present first and second options that are selectable by the patient, wherein the first option, when selected by the patient, allows the patient to choose the subset of doctors from the first set of doctors, and wherein the second option, when selected by the patient, allows the server to automatically choose the subset of doctors from the first set of doctors.

14. The system of claim 11, wherein the server is further configured to receive, from the patient device, a set of preferences provided by the patient, and wherein the availability request is communicated to the subset of doctors such that each doctor of the subset of doctors satisfies the set of preferences.

15. The system of claim 14, wherein the set of preferences includes a type of doctor, a timespan of medical experience of the doctor, or a review rating threshold of the doctor.

16. The system of claim 11, wherein the server is further configured to:
receive, from a plurality of doctor devices, a plurality of credential proofs of a plurality of doctors,
verify credentials of each doctor of the plurality of doctors based on the plurality of credential proofs, and
register the plurality of doctors based on the verification of the corresponding credentials, wherein the first set of doctors is identified from the registered plurality of doctors.

17. The system of claim 11, wherein the server is further configured to communicate patient details of the patient to the first doctor based on the selection of the first doctor, and wherein the patient details are indicative of contact information and a medical condition of the patient.

18. The system of claim 11, wherein the server is further configured to render, on the patient device, a graphical interface to present a profile of each of the identified first set of doctors to the patient.

19. The system of claim 18, wherein the profile of each doctor includes at least one of a name of the corresponding doctor, a timespan of medical experience of the corresponding doctor, a specialization of the corresponding doctor, or a review rating of the corresponding doctor.

20. A non-transitory computer-readable storage medium having thereon, computer executable instructions, which when executed by a computer, cause the computer to execute operations, the operations comprising:
identifying based on a first location of a patient, a first set of doctors for performing a medical procedure;
communicating an availability request to a subset of the first set of doctors, requesting confirmation of an availability of each doctor of the subset of the first set of doctors for performing the medical procedure;
receiving, from one or more doctor devices, one or more availability responses of one or more doctors, wherein each availability response is indicative of an availability of a corresponding doctor for performing the medical procedure, and wherein the subset of doctors comprises the one or more doctors;
selecting a first doctor from the one or more doctors, for performing the medical procedure, wherein the first doctor is selected based on a response time of the availability response of the first doctor to the availability request; and
communicating, to a patient device of the patient, a notification indicating the selection of the first doctor for performing the medical procedure.
